(19) United States
(12) Patent Application Publication
(10) Pub. No.: US 2022/0409533 A1
(43) Pub. Date: Dec. 29, 2022

(54) LAG TIME REDUCTION/ICE SPRAY

(71) Applicant: LTS LOHMANN THERAPIE-SYSTEME AG, Andemach (DE)

(72) Inventors: James Paul RONNANDER, Mt. Vernon, NY (US); Andreas KOCH, Melsbach (DE)

(21) Appl. No.: 17/773,383

(22) PCT Filed: Sep. 28, 2020
(86) PCT No.: PCT/EP2020/077108

§ 371 Date: Apr. 29, 2022

(30) Foreign Application Priority Data
Oct. 31, 2019 (DE) 10 2019 129 444.4

(51) Int. Cl. A61K 9/12; A61K 9/00; A61K 31/381; A61K 31/5386; A61K 31/196

(52) U.S. Cl. CPC A61K 9/124; A61K 9/0014; A61K 31/381; A61K 31/5386; A61K 31/196

(57) ABSTRACT

The present invention relates to a kit, comprising at least one cold spray and at least one dosage form for transdermally administering at least one pharmaceutically active ingredient, to said kit for use in the treatment of a patient, to the use of a cold spray in order to shorten the resorption delay time in the application of a dosage form for transdermally administering at least one pharmaceutically active ingredient, and to a method for treating a patient.

The present invention relates to a kit, comprising at least one cold spray and at least one dosage form for transdermally administering at least one pharmaceutically active ingredient, to said kit for use in the treatment of a patient, to the use of a cold spray in order to shorten the resorption delay time in the application of a dosage form for transdermally administering at least one pharmaceutically active ingredient, and to a method for treating a patient, wherein the skin of the patient is firstly cooled in at least one area and a dosage form for transdermally administering at least one pharmaceutically active ingredient is then applied to the cooled area of the skin.

Dosage forms for transdermally administering at least one pharmaceutically active ingredient have enjoyed widespread use in recent years in the treatment of numerous diseases since they are associated with certain advantages as compared to other administration forms.

On the one hand, for example, the stomach, intestine and liver are protected due to an avoidance of the gastrointestinal tract. On the other hand, the first-pass effect can be bypassed and the compliance can be increased, since the patient does not have to take tablets at regular intervals. In comparison to oral administration forms such as tablets, it is also possible to achieve a continuous and controlled release of the active ingredient over a longer period of time, without a risk of overdosing or underdosing.

Dosage forms for transdermally administering at least one pharmaceutical active ingredient have the disadvantage, however, that depending on the active ingredient there is a so-called resorption delay time until the onset of a systemic effect (lag time).

This is due to the fact that the transdermally applied active ingredient must first reach or diffuse to the site of its resorption; in this case, the pathway from the interface between the dosage form and stratum corneum as the uppermost or outermost skin layer to the stratum papillare, in which the first blood vessels (superficial blood vessel network) are located as the organ of resorption.

The resorption delay time (lag time) is therefore the time from which a uniform diffusion flow of the active ingredient from the dosage form and thus the onset of the therapeutic effect occurs. Depending on the diffusion speed of the active ingredients concerned, this lag time can range from 6 hours, as in the case of nicotine for example, to 24 hours, as in the case of buprenorphine.

In the case of buprenorphine, for example, the relatively long resorption delay time (lag time) means that a pain patient who is to be switched from oral buprenorphine tablets to a transdermal application of buprenorphine must receive a bolus injection at the same time as the transdermal application so that he or she is well supplied with pain therapy until the onset of the systemic effect through the transdermal application. However, the quickest possible onset of action is also desired for other indications, such as the treatment of dizzy spells or migraine attacks.

Methods for shortening the resorption delay time in the application of dosage forms for transdermally administering a pharmaceutically active ingredient are known from the prior art.

For example, WO 2005/100603 A2 discloses a pre-treatment of the skin by tape stripping (removal of the uppermost cell layers of the stratum corneum) and WO 96/34566 A1 discloses a pre-treatment of the skin by a laser.

However, these known methods for pre-treatment of the skin have the disadvantage that the skin is irreversibly damaged in the process.

The aim of the present invention was to shorten the resorption delay time (lag time) in the application of dosage forms for transdermally administering at least one pharmaceutically active ingredient, so that a faster constant flow of active ingredient (=faster onset of action) can be achieved without causing lasting damage to the skin.

This aim was surprisingly addressed by the use of a cold spray to shorten the resorption delay time (lag time) in the application of a dosage form for transdermally administering at least one pharmaceutically active ingredient.

It has been found that the skin barrier can be reduced by treatment with a cold spray and thus the resorption delay time (lag time) in the subsequent application of a dosage form for transdermally administering at least one pharmaceutically active ingredient can be shortened.

The present invention therefore relates to a kit comprising at least one cold spray and at least one dosage form for transdermally administering at least one pharmaceutically active ingredient.

Hereinafter, the word “comprising” can also mean “consisting of”.

In the context of the present invention, the terms “resorption delay time” and “lag time” are used synonymously.

In the context of the present invention, the terms “cold spray” and “ice spray” are also used synonymously.

A cold spray is understood to mean a gas that is filled in a spray can and that is used to cool objects or parts of the body. With such known cold sprays, temperatures of −25 to −55° C. are preferably achieved. Preferably, different gases and gas mixtures are present in the cold sprays, with the lowest achievable temperature and flammability being the main distinguishing criteria.

The at least one dosage form for transdermally administering at least one pharmaceutically active ingredient is in principle not restricted.

The kit according to the invention is preferably characterised in that the at least one dosage form comprises a transdermal therapeutic system, a gel, a lotion, an ointment, a cream and/or a microneedle system.

Especially preferably, the kit according to the invention is characterised in that the at least one dosage form comprises a transdermal therapeutic system.

A transdermal therapeutic system (also called a transdermal patch) is understood to mean a system to be applied to the skin, preferably a patch, with a defined application area, which can deliver a pharmaceutically active ingredient to a patient's body in a controlled manner, preferably according to time and quantity.

Such systems usually have a cover film (backing layer) that protects the patch and its contents from the outside and may be printed with information. Towards the skin side, a peel-off film (release liner) is preferably provided, which covers the sticky side of the system. The peel-off film is removed before the system is applied and is often siliconised to facilitate removal.

With regard to the technique of controlled drug delivery from the system, a distinction can be made between matrix systems (matrix patches) and membrane systems (also called reservoir or depot systems or reservoir or depot patches).

In matrix systems, the active ingredient is contained in a matrix which consists of one or more layers and which is applied directly to the skin with the aid of an adhesive layer. Embodiments are also possible in which the matrix is simultaneously the adhesive layer. The diffusion rate of the active ingredient out of the matrix determines the resorption rate.

In some embodiments, there may be an additional membrane between the matrix and adhesive layers that controls the flow of active ingredient.

In the membrane systems, a reservoir of the active ingredient lies under a carrier film, and the active ingredient is released from the reservoir through a porous membrane into the skin in a controlled manner.

The advantages of a transdermal therapeutic system on the patient side are a safe, reliable, exact and painless dosage of pharmaceutically active ingredients and the easier therapy of children, elderly patients and patients in need of care. Furthermore, transdermal therapeutic systems are ideal for patients with swallowing difficulties and for extended dosing intervals, especially with multi-day patches.

The advantages of a transdermal therapeutic system on the manufacturer's side are the possible formulation of pharmaceutically active ingredients with only low oral bioavailability, a controlled, uniform delivery of pharmaceutically active ingredients without active ingredient peaks, a good possibility to control the drug dosage by varying the area, no loss of active ingredient by avoiding first-pass metabolism in the liver and no degradation of the active ingredient in the gastrointestinal tract.

Gels usually comprise gelled liquids. They are preferably produced with suitable swelling agents (gelling agents). These include, for example, celluloses, starches, carbomers, gelatine, xanthan, bentonite, agar and/or pectin.

A distinction is made here between hydrophilic and lipophilic gels. Gels can be transparent or opaque.

Other possible ingredients include water, propylene glycol, antioxidants, lipids (in lipogels), flavourings, sweeteners and/or preservatives.

Among other things, gels are used for the local or systemic administration of active ingredients and for moist wound treatment.

A lotion is an externally applied liquid aqueous or aqueous-alcoholic preparation containing suspended or emulsified pharmaceutically active ingredients, and possibly excipients.

Lotions are generally more liquid than creams or ointments and are therefore easier to apply to large areas of the skin.

A lotion is an externally applied oil-in-water emulsion or water-in-oil emulsion. It is very light and does not lubricate.

Lotions are used, among other things, for the local or systemic administration of active ingredients and for moist wound treatment.

An ointment, preferably a suspension ointment, is a semi-solid preparation for external use. Ointments preferably consist of a single-phase base in which solid or liquid substances may be dispersed.

A distinction is made between hydrophobic ointments, water-absorbing ointments and hydrophilic ointments. Ointments can also contain emulsifiers and water.

For example, fatty oils, fats, waxes, petroleum products such as petrolatum and paraffins, triglycerides and/or macrogols (PEG) can be used to make ointments.

Ointments are used, among other things, for the local or systemic administration of active ingredients and for moist wound treatment.

A cream is a semi-solid preparation, usually for application to the skin.

A cream is preferably a multi-phase preparation consisting of a lipophilic and an aqueous phase and containing at least one pharmaceutically active ingredient. A distinction is made between a hydrophilic cream (oil-in-water) and a lipophilic/hydrophobic cream (water-in-oil).

A cream is used, among other things, for the local or systemic administration of active ingredients and for moist wound treatment.

A microneedle system, also called a microneedle array, is a system comprising a plurality of microneedles on a carrier. The needles, which preferably have a length of 100 μm to 1000 μm, can be made of different materials such as ceramics, steel, polymers or silicon. In this context, the pharmaceutically active agent can on the one hand be administered through micropores, and on the other hand biodegradable polymers can be used in the needles and thus the pharmaceutically active agent can be administered by dissolving the needles. Microneedle systems are described, for example, in WO 2019/115815 A1 or WO 2016/162449 A1.

The at least one pharmaceutically active ingredient is in principle not restricted. Preferably, the at least one pharmaceutically active ingredient comprises an active ingredient for indications in which the fastest possible onset of action is desired.

The kit according to the invention is characterised in that the at least one pharmaceutically active ingredient is selected from the group consisting of hypnotics, sedatives, antiepileptics, analeptics, psychoneurotropic drugs, neuromuscular blockers, antispasmodics, antihistamines, antiallergics, cardiotonics, antiarrhythmics, diuretics, hypotensives, vasopressors, antitussives, expectorants, analgesics, thyroid hormones, sexual hormones, glucocorticoid hormones, antidiabetics, antitumour drugs, antibiotics, chemotherapeutics, narcotics, anti-Parkinson drugs, anti-Alzheimer drugs and/or triptans.

The pharmaceutically active ingredient is especially preferably selected from the group consisting of antiepileptics, antihistamines, antiallergics, analgesics or triptans. For these indications especially, the quickest possible onset of action plays an important role.

The kit according to the invention is preferably characterised in that the cold spray comprises a liquid gas filled in a spray can.

The liquid gas preferably comprises propane, butane, pentane, tetrafluoroethane, dimethyl ether and/or chloroethane.

An expedient cold spray is, for example, Servisol® Freeze IT 20 from Servisol, Bridgwater, UK, based on a mixture of tetrafluoroethane and dimethyl ether.

The present invention further relates to the kit described above for use in treating a patient.

The present invention also relates to the use of a cold spray to shorten the resorption delay time (lag time) in the application of a dosage form for transdermally administering at least one pharmaceutically active agent.

The resorption delay time (lag time), as described above, means the time from which a uniform diffusion flow of the active ingredient from the dosage form for transdermally administering at least one pharmaceutically active ingredient and thus the onset of the therapeutic effect occurs.

The definitions and advantageous embodiments set out above with regard to the kit with the associated advantages apply analogously to the use of a cold spray according to the invention.

The use of a cold spray according to the invention is preferably characterised in that the dosage form comprises a transdermal therapeutic system, a gel, a lotion, an ointment and/or a microneedle system.

The use of a cold spray according to the invention is preferably characterised in that the at least one pharmaceutically active ingredient comprises an active ingredient for indications in which the fastest possible onset of action is desired.

The use of a cold spray according to the invention is preferably characterised in that the at least one pharmaceutically active ingredient is selected from the group consisting of hypnotics, sedatives, antiepileptics, analeptics, psychoneurotropic drugs, neuromuscular blockers, antispasmodics, antihistamines, antiallergics, cardiotonics, antiarrhythmics, diuretics, hypotensives, vasopressors, antitussives, expectorants, analgesics, thyroid hormones, sexual hormones, glucocorticoid hormones, antidiabetics, antitumour drugs, antibiotics, chemotherapeutics, narcotics, anti-Parkinson drugs, anti-Alzheimer drugs and/or triptans.

The pharmaceutically active ingredient is especially preferably selected from the group consisting of antiepileptics, antihistamines, antiallergics, analgesics or triptans.

In a preferred embodiment the cold spray is characterised in that the cold spray comprises a liquid gas filled in a spray can.

The cold spray preferably comprises propane, butane, pentane, tetrafluoroethane, dimethyl ether and/or chloroethane.

The present invention also relates to a method for treating a patient comprising the steps of:
cooling at least one area of the patient's skin and
applying a dosage form for transdermally administering at least one pharmaceutically active ingredient to the cooled area of the skin.

The cooling causes a reduction of the skin barrier and thus a shortened resorption delay time. The cooling is reversible. Reversible means that especially no freezer burn (gentle cooling) and especially no macroscopic skin damage is produced.

The method according to the invention is preferably characterised in that the patient's skin is cooled by being treated with a cold spray.

In this context, the above definitions and preferred embodiments for the cold spray also apply analogously to the method according to the invention.

The method according to the invention is preferably characterised in that the treatment with a cold spray is carried out for 3 sec to 20 sec, preferably for 5 sec to 15 sec, Especially preferably for at most 10 sec. This ensures that the reduction of the skin barrier is reversible.

The method according to the invention is preferably characterised in that less than 15 sec, preferably less than 10 sec, elapse between the cooling (cold treatment) of at least one area of the patient's skin and the application of a dosage form for transdermally administering at least one pharmaceutically active ingredient to the cooled area of the skin. If this time is exceeded, the beneficial effect of reducing the skin barrier is no longer sufficiently provided.

The method according to the invention is preferably characterised in that the distance of the spray head of the cold spray from the patient's skin is at least 15 cm, preferably 15 cm to 30 cm. This ensures that the patient's skin is not irreversibly damaged.

Especially preferably, the treatment is carried out with a cold spray for a maximum of 10 s and with a distance of at least 15 cm between the spray head of the cold spray and the patient's skin.

The method according to the invention is preferably characterised in that the dosage form comprises a transdermal therapeutic system, a gel, a lotion, a suspension ointment and/or a microneedle system.

In this context, that described above applies analogously to the method according to the invention with regard to the individual dosage forms.

The method according to the invention is preferably characterised in that the cooling of a patient before applying a dosage form for transdermally administering at least one pharmaceutically active ingredient to the cooled area of the skin causes the resorption delay time to be shortened by at least a factor of 1.3, preferably by at least a factor of 1.4, especially preferably by at least a factor of 1.7 and very Especially preferably by at least a factor of 2.0.

### FIGURES

FIG. 1 shows the results of the measurement of the in vitro human skin permeation during the application of test samples of the transdermal therapeutic system Neupro® (10 cm² TTS from UCB Pharma GmbH, Germany, with a delivery rate of 2 mg/24 h and a content of 4.5 mg active ingredient, pharmaceutical registration number 10393704) for the administration of rotigotine with and without a prior skin treatment with a cold spray. The resorption delay time was reduced by a factor of 1.4.

FIG. 2 shows the results of the measurement of the in vitro human skin permeation during the application of test samples of the transdermal therapeutic system Scopoderm® (2.5 cm² TTS from Novartis Consumer Health GmbH, Germany, with a delivery rate of 1.0 mg/72 h and a content of 1.54 mg active ingredient, pharmaceutical registration number 00107146) for the administration of scopolamine with and without a prior skin treatment with a cold spray. The resorption delay time was reduced by a factor of 2.0.

FIG. 3 shows the results of the measurement of in vitro human skin permeation during the application of the transdermal therapeutic system Voltaren Tape 30 Dojin® (140 cm² patch from Iyaku-Kako Co Ltd, Japan, with a content of 30 mg active ingredient; a delivery rate cannot be given for this system as it is a topical system. As the product is only approved and distributed in the Asian region, no pharmaceutical registration number can be given). for the administration of Diclofenac sodium with and without a prior skin treatment with a cold spray. The resorption delay time was reduced by a factor of 2.0.

The invention will be described in greater detail hereinafter on the basis of non-limiting examples.

### EXAMPLE 1

The reduction of the skin barrier was determined by measuring the TEWL (transepidermal water loss). The higher the TEWL value, the more the skin barrier is reduced.

For this purpose, TEWL values were determined using an AquaFlux200 measuring probe from Biox, London, UK. The sample in each case was 800 μm dermatomised human abdominal skin from a 71-year-old female donor who was treated for a certain time with the cold spray Servisol® Freeze IT 20 (Servisol Bridgewater, Somerset, UK) from a distance of about 15 cm. The measurement results before and after cold spray treatment are summarised in Table 1.

TABLE 1

| | | TEWL | TEWL | | |
|---|---|---|---|---|---|
| | | before cold | after cold | TEWL | TEWL after |
| Test | Treatment | treatment | treatment | increase | 30 min |
| no. | type | [g/m² × h] | [g/m² × h] | [g/m² × h] | [g/m² × h] |
| 1 | 5 sec cold | 15.63 | 46.90 | 31.28 | 14.71 |
| | spray | | | | |
| 2 | 5 sec cold | 14.91 | 52.29 | 37.38 | 18.74 |
| | spray | | | | |
| 3 | 10 sec cold | 13.54 | 56.84 | 43.30 | 16.42 |
| | spray | | | | |
| 4 | 10 sec cold | 14.44 | 47.73 | 32.99 | 13.15 |
| | spray | | | | |

According to the German Society for Dermatopharmacy, an intact skin barrier is characterised by a TEWL range of 10 to 24 g/m²×h. The values in the sixth column show that the process is reversible and does not lead to skin damage.

### EXAMPLE 2

The in vitro human skin permeation of three commercially available transdermal therapeutic systems without and with prior cold treatment was determined. For this purpose, the skin samples were treated completely over their surface for 10 sec from a distance of about 15 cm with a cold spray (Servisol® Freeze IT 20). After about 10 sec (an ice film is formed, which should preferably have disappeared again first), the corresponding transdermal therapeutic system was applied to the treated area of the skin and the permeation profile (cumulatively permeated amount as a function of time) was measured using a Franz cell.

The donor compartment of the Franz cell contained the transdermal therapeutic system. The acceptor compartment of the Franz cell was filled with buffer or other solutions. By regularly taking samples from the acceptor compartment, the permeation of a substance through the skin could be followed over the selected time period. The use of the Franz cell as a diffusion model is especially suitable for predicting the transport of drugs through human skin (=permeation), which corresponds to systemic availability.

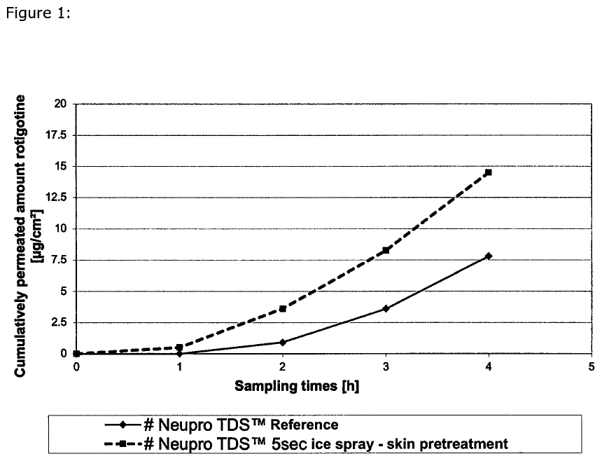

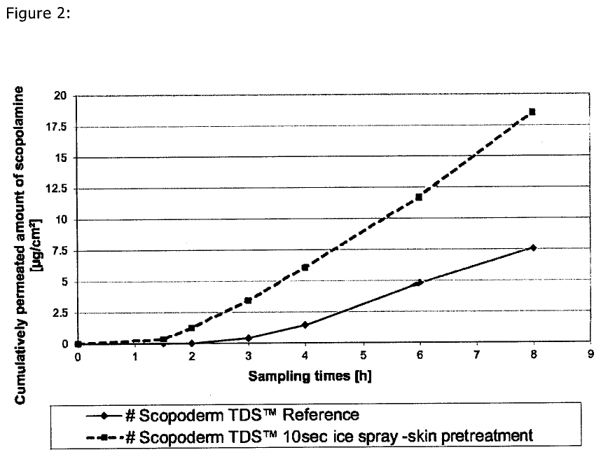

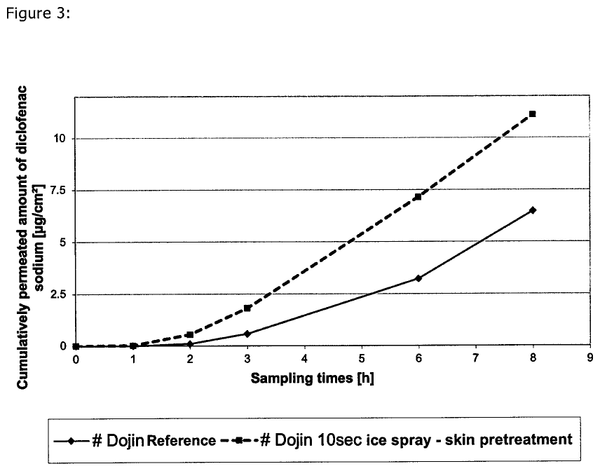

Here, circular die-cuts, measuring 1.17 cm², of the transdermal therapeutic systems were glued onto the epidermal side (skin surface) of 800 μm dermatomised circular skin pieces with a diameter of 25 mm, once after and once without (reference) skin pre-treatment. An aqueous isotonic phosphate buffer pH=5.5 plus 0.1% sodium azide as preservative with a filling volume of 10 ml was used as acceptor medium. The permeation was measured at a temperature of 32° C. and the values were determined after 1, 2, 3 and 4 hours for Neupro®, after 1.5, 2, 3, 4, 6 and 8 hours for Scopoderm® and after 1, 2, 3, 6 and 8 hours for Dojin®. The respective measurement results with the transdermal therapeutic systems Neupro®, Scopoderm® and Dojin® can be seen in FIGS. 1, 2 and 3.

1. A kit, comprising at least one cold spray and at least one dosage form for transdermally administering at least one pharmaceutically active ingredient.

2. The kit according to claim 1, characterised in that the at least one dosage form comprises a transdermal therapeutic system, a gel, a lotion, an ointment, a cream and/or a microneedle system.

3. The kit according to claim 1, characterised in that the at least one pharmaceutically active ingredient is selected from the group consisting of hypnotics, sedatives, antiepileptics, analeptics, psychoneurotropic drugs, neuromuscular blockers, antispasmodics, antihistamines, antiallergics, cardiotonics, antiarrhythmics, diuretics, hypotensives, vasopressors, antitussives, expectorants, analgesics, thyroid hormones, sexual hormones, glucocorticoid hormones, antidiabetics, antitumour drugs, antibiotics, chemotherapeutics, narcotics, anti-Parkinson drugs, anti-Alzheimer drugs and/or triptans.

4. The kit according to claim 1, characterised in that the cold spray comprises a liquid gas filled in a spray can.

5. The kit according to claim 4, characterised in that the liquid gas comprises propane, butane, pentane, tetrafluoroethane, dimethyl ether and/or chloroethane.

6. The kit according to claim 1 for use in the treatment of a patient.

7. A method for reducing the resorption delay time in the application of a dosage form comprising transdermally administering at least one pharmaceutically active ingredient in a cold spray.

8. The method according to claim 7, characterised in that the dosage form comprises a transdermal therapeutic system, a gel, a lotion, a suspension ointment and/or a microneedle system.

9. The method according to claim 7, characterised in that the at least one pharmaceutically active ingredient is selected from the group consisting of hypnotics, sedatives, antiepileptics, analeptics, psychoneurotropic drugs, neuromuscular blockers, antispasmodics, antihistamines, antiallergics, cardiotonics, antiarrhythmics, diuretics, hypotensives, vasopressors, antitussives, expectorants, analgesics, thyroid hormones, sexual hormones, glucocorticoid hormones, antidiabetics, antitumour drugs, antibiotics, chemotherapeutics, narcotics, anti-Parkinson drugs, anti-Alzheimer drugs and/or triptans.

10. The method according to claim 7, characterised in that the cold spray comprises a liquid gas filled in a spray can.

11. The method according to claim 10, characterised in that the liquid gas comprises propane, butane, pentane, tetrafluoroethane, dimethyl ether and/or chloroethane.

12. A method for treating a patient, comprising the steps of
cooling at least one area of the patient's skin and
applying a dosage form for transdermally administering at least one pharmaceutically active ingredient to the cooled area of the skin.

13. The method according to claim 12, characterised in that the skin is cooled by being treated with a cold spray.

14. The method according to claim 13, characterised in that the treatment with a cold spray lasts for between 3 sec and 20 sec.

15. The method according to claim 12, characterised in that the dosage form comprises a transdermal therapeutic system, a gel, a lotion, a suspension ointment and/or a microneedle system.
